(19) United States
(12) Patent Application Publication
(10) Pub. No.: US 2003/0232881 A1
(43) Pub. Date: Dec. 18, 2003

(54) Crystals of pharmaceutically acceptable salts of citalopram, methods of crystallization, and pharmaceutical compositions comprising them

(75) Inventors: Ken Liljegren, Vaerlose (DK); Per Holm, Vanlose (DK); Ole Nielsen, Valby (DK); Sven Wagner, Sodertalje (SE)

Correspondence Address: DARBY & DARBY P.C., 805 Third Avenue, New York, NY, 10022, US

(73) Assignee: H. LUNDBECK A/S, Valby-Copenhagen (DK)

(21) Appl. No.: 10/310,621

(22) Filed: Dec. 5, 2002

Related U.S. Application Data
(63) Continuation-in-part of application No. 09/730,380, filed on Dec. 5, 2000.

(30) Foreign Application Priority Data
Oct. 27, 2000 (DK) PA 2000 01614

(51) Int. Cl. C07D 307/87; A61K 31/343

(52) U.S. Cl. 514/469; 549/467

(57) ABSTRACT

The invention is directed to methods of crystallizing pharmaceutically acceptable salts of citalopram, the resulting crystals, and pharmaceutical compositions comprising the crystals.

This application is a continuation in part of U.S. patent application Ser. No. 09/730,380, filed Dec. 5, 2000.

The present invention is directed to crystals of pharmaceutically acceptable salts of citalopram, 1-[3-(dimethylamino)propyl]-1-(4-fluorophenyl)-1,3-dihydro-5-isobenzo-furancarbonitrile, methods of crystallization, and pharmaceutical compositions comprising the crystals.

### BACKGROUND OF THE INVENTION

Citalopram is a well-known antidepressant drug that has the following structure:

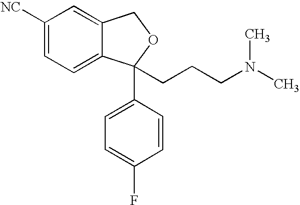

Citalopram is a selective, centrally active serotonin (5-hydroxytryptamine; 5-HT) reuptake inhibitor, accordingly having antidepressant activities.

Citalopram was first disclosed in DE 2,657,013 which corresponds to U.S. Pat. No. 4,136,193. DE '013 describes the preparation of citalopram by one method and outlines a further method, which may be used for preparing citalopram. Citalopram was prepared and isolated in crystalline form as the oxalate, the hydrobromide and the hydrochloride salt, respectively. Furthermore, citalopram base was obtained as an oil (B.P. 175° C./0.03 mmHg). DE 013 also outlines the manufacture of tablets containing salts of citalopram.

Manufacture of crystalline citalopram base is disclosed in co-pending DK 2000 00402, This patent publication describes the preparation of crystalline citalopram base and the use of crystalline citalopram base as an intermediate in the purification of crude citalopram hydrobromide into pure citalopram hydrobromide. The publication also outlines the manufacture of tablets containing citalopram base.

Citalopram is marketed in a number of countries as a tablet prepared by compression of granulated citalopram hydrobromide, lactose and other excipients.

It is well recognised that preparation of tablets with a reproducible composition requires that all the dry ingredients have good flow properties. In cases where the active ingredient has good flow properties, tablets can be prepared by direct compression of the ingredients. However, in many cases the particle size of the active substance is small, the active substance is cohesive or has poor flow properties. Further, active substances with a small particle size mixed with excipients having a larger particle size will typically segregate or de-mix during the tabletting process.

The problem of small particle size and poor flowability, is conventionally solved by enlarging the particle size of the active substance, usually by granulation of the active ingredient either alone or in combination with a filler and/or other conventional tablet ingredients.

One such granulation method is the “wet” granulation process. Using this method, the dry solids (active ingredients, filler, binder etc.) are blended and moistened with water or another wetting agent (e.g. an alcohol) and agglomerates or granules are built up of the moistened solids. Wet massing is continued until a desired homogenous particle size has been achieved whereupon the granulated product is dried.

An alternative to the “wet” granulation method is the “melt” granulation method, which is also known as the “thermal plastic” granulation process, where a low melting solid is used as the granulation agent. Initially, the dry solids are blended and heated until the binder melts. As the binder is liquefied and spreads over the surface of the particles, the particles adhere to each other and form granules. The binder solidifies upon cooling to form a dry granular product.

Wet granulation as well as melt granulation are energy intensive unit operations requiring complicated and expensive equipment as well as technical skill.

The process used for the preparation of citalopram hydrobromide results in a product with a very small particle size (around 2-20 μm) that has very poor flow properties. Thus, in order to achieve appropriate dosing of the citalopram during tabletting, it was considered necessary to make a granulate of citalopram with larger particle size and improved flow properties. The citalopram tablet that is marketed is a tablet made from granulated citalopram hydrobromide with various excipients.

In view of the fact that direct compression is much simpler and cheaper than the processes involving granulation there is a desire for a process for direct compression of citalopram hydrobromide.

The obstacles that hitherto have hindered direct compression of citalopram tablets have now been circumvented after extensive laboratory research.

It has been found that larger particles, i.e. particles of a size comparable to the size of the filler, may be prepared by a new and inventive crystallisation process and that these particles are useful for the manufacture of directly compressed tablets. Accurate dosing in capsules may also be with such large particles.

### OBJECTS OF THE INVENTION

One object of the invention is to provide large crystals of a pharmaceutically acceptable salt of citalopram suitable for use in direct compression.

Another object of the invention is to provide a method for crystallization of large crystals of a pharmaceutically acceptable salt of citalopram.

It is another object of the invention to provide a novel pharmaceutical unit dosage form containing citalopram with a suitable large particle size, wherein said unit dosage form may be prepared by direct compression.

### SUMMARY OF THE INVENTION

The invention is directed in one embodiment to crystals of a pharmaceutically acceptable salt of citalopram, suitable for use in a solid unit dosage form, with a median particle size of at least 40 μm. Crystals of a pharmaceutically acceptable salt of citalopram of the invention may have the following properties:

(1) a particle size of less than 5 μm in a proportion of 35% at most.

(2) a particle size of less than 5 μm in a proportion of 35% at most, and containing crystals having a particle size of not less than 20 μm in a proportion of not less than 10%, or having an average aspect ratio of not less than 2.0 and not more than 9.0, or having an average aspect ratio of not less than 2.5 and less than 4.5, or having an average aspect ratio of not less than 4.5 and not more than 6.0.

(3) an average aspect ratio of not less than 2.0 and not more than 9.0.

(4) an average aspect ratio of not less than 2.5 and less than 4.5.

(5) an average aspect ratio of not less than 4.5 and not more than 6.0.

Crystals of a pharmaceutically acceptable salt of citalopram containing crystals having a particle size of less than 5 μm in a proportion of 35% at most and crystals of a pharmaceutically acceptable salt of citalopram having an average aspect ratio of not less than 4.5 and not more than 6.0, which are obtained by this method, are advantageous as a pharmaceutical bulk.

The preferred pharmaceutically acceptable salt of citalopram is citalopram hydrobromide.

The invention is also directed to solid unit dosage forms comprising a pharmaceutically acceptable salt of citalopram. Sample unit dosage forms of the invention are prepared by direct compression of a mixture of citalopram base or a pharmaceutically acceptable salt thereof and pharmaceutically acceptable excipients, or by filling of said mixture in a hard gelatine capsule.

The invention is also directed to methods for the manufacture of crystals of a pharmaceutically acceptable salt of citalopram having a median particle size of at least 40 μm and suitable for use in a solid unit dosage form wherein a solution of a pharmaceutically acceptable salt of citalopram is dissolved, for example by heating, in a suitable solvent system at a first temperature. The solvent may comprise at least one member selected from the group consisting of one or more alcohols having 1 to 3 carbon atoms, water and acetone The solution is first cooled down to a second temperature, then seeded by addition of crystals of said citalopram salt followed optionally by a holding time at said second temperature and a controlled cooling down to a third temperature whereupon said crystals are isolated by conventional solid/liquid separation techniques.

In another embodiment of the invention for crystallizing a pharmaceutically acceptable salt of citalopram, the step of crystallizing may also comprise, after the step of crystallization, repeating the step of heating, to dissolve a part of the obtained crystals.

Definitions

As used herein, “direct compression” means that the solid unit dosage form is prepared by compression of a simple mixture of the active ingredient and excipients, without the active ingredient having been subjected to an intermediate granulation process in order to embed it in a larger particle and improve its fluidity properties.

As used herein, “binder” means an agent, which is used in wet or melt granulation processes and acts as a binder in the granulated product.

As used herein, “particle size distribution” means the distribution of equivalent spherical diameters as determined by laser diffraction at 1 bar dispersive pressure in a Sympatee Helos equipment. “Median particle size”, correspondingly, means the median of said particle size distribution.

As used herein, “refluxing temperature” means the temperature at which the solvent or solvent system refluxes or boils at atmospheric pressure.

As used herein, the “average particle size” means the average diameter upon conversion of the volume of the crystal particles into sphere.

As used herein, the “average aspect ratio” means the average value of the major axis/minor axis of crystal particles.

### DETAILED DESCRIPTION OF THE INVENTION

Citalopram Salt Crystals

In one embodiment, the invention is directed to crystals of a pharmaceutically acceptable salt of citalopram, having a median particle size of greater than 4 μm, preferably in the range of 40-200 μm, preferably 45-150 μm and even more preferred 50-120 μm.

In a preferred embodiment of the present invention the crystals are of citalopram hydrobromide or citalopram hydrochloride, most preferably citalopram hydrobromide.

Crystals of a pharmaceutically acceptable salt of citalopram of the invention may have the following properties:

(1) a particle size of less than 5 μm in a proportion of 35% at most.

(2) a particle size of less than 5 μm in a proportion of 35% at most, and containing crystals having a particle size of not less than 20 μm in a proportion of not less than 10%, or having an average aspect ratio of not less than 2.0 and not more than 9.0, or having an average aspect ratio of not less than 2.5 and less than 4.5, or having an average aspect ratio of not less than 4.5 and not more than 6.0.

(3) an average aspect ratio of not less than 2.0 and not more than 9.0.

(4) an average aspect ratio of not less than 2.5 and less than 4.5.

(5) an average aspect ratio of not less than 4.5 and not more than 6.0.

Crystals of a pharmaceutically acceptable salt of citalopram containing crystals having a particle size of less than 5 μm in a proportion of 35% at most and crystals of a pharmaceutically acceptable salt of citalopram having an average aspect ratio of not less than 4.5 and not more than 6.0, which are obtained by this method, are advantageous as a pharmaceutical bulk.

Exemplary particle size distribution for the crystals of the invention include: less than 5 μm: 17.1%, not less than 5 μm and less than 10 μm: 22.2%; not less than 10 μm and less than 20 μm: 29.1%, not less than 20 μm and less than 40 μm: 27.0%; not less than 40 μm 4.6%; average particle size 12.9 μm; and average aspect ratio: 4.8.

Another example of particle size distribution according to the invention is less than 5 μm: 9.6%; not less than 5 μm and less than 10 μm: 12.3%; not less than 10 Am and less than 20 μm: 20.9%; not less than 20 μm and less than 40 μm: 31.7%; not less than 40 μm: 25.5%; average particle size: 23.8 μm; and average aspect ratio: 5.4.

Another example of particle size distribution according to the invention is less than 5 μm: 12.6%; not less than 5 μm and less than 10 μm: 20.2%; not less than 10 μm and less than 20 μm: 35.5%; not less than 20 μm and less than 40 μm: 29.3%; not less than 40 μm: 2.4%; average particle size: 14.6 μm; and average aspect ratio: 3.0

According to the method of the present invention, the crystal characteristics of the pharmaceutically acceptable salt of citalopram, such as particle size, particle size distribution, aspect ratio and the like, can be controlled easily and in an industrial setting. In addition, citalopram hydrobromide crystals having crystal characteristics useful as a pharmaceutical bulk can be provided.

Method of Crystallizing Pharmaceutically Acceptable Salts of Citalopram

The invention is also directed to methods of crystallizing the aforementioned crystals of a pharmaceutically acceptable salt of citalopram. According to the method of the invention, the citalopram salt is crystallized from a solvent system comprising one or more alcohols and optionally water. Preferably the solvent system is a mixture of methanol and water, wherein the methanol: water weight ratio preferably is in the range of 5:1 to 50:1.; even more preferred 10:1 to 30:1 and most preferred 15:1 to 25:1.

The pharmaceutically acceptable salt of citalopram is preferably dissolved in the solvent system at a temperature in the range between 50° C. and the refluxing temperature of the solvent system, preferably between 60° C. and the refluxing temperature, and more preferably between 64° C. and the refluxing temperature. The amounts of pharmaceutically acceptable salt of citalopram and solvent used preferably correspond to a solvent: solute weight ratio in the range of 0.5:1 to 5:1, more preferred 0.7:1 to 2:1 and most preferred 0.9:1 to 1.5:1.

In the method of the invention, the solution of a pharmaceutically acceptable salt of citalopram is cooled down to a temperature, the seeding temperature, in the range of 20-40° C., preferably 25-35° C., whereupon it is seeded with citalopram crystals and optionally kept at said seeding temperature for a holding time for crystal growth in the range of 30 minutes to 7 days, preferably 1 hour to 4 days, and more preferably 12 to 36 hours. After said holding time, the crystallisation batch is gradually cooled down in a controlled way from the seeding temperature to the temperature at which the crystals will be isolated from the mother liquor, wherein said gradual cooling down is done over a time span in the range of 5 minutes to 6 hours, preferably 15 minutes to 4 hours and more preferred 30 minutes to 2 hours. The crystals of said pharmaceutically acceptable salt of citalopram are preferably isolated from the mother liquor at a temperature in the range of 0-20° C., more preferred 5-15° C., using conventional separation techniques, e.g. filtration.

In the method of the invention, the step of dissolving may also include stirring, e.g., stirring at 10 rpm-250 rpm, particularly at 50 rpm-220 rpm. Moderate stirring is preferred.

In the method of the invention, the pharmaceutically acceptable salt of citalopram is dissolved in a specific solvent system preferably by heating to from 50° C. to the reflux temperature of the solvent system, e.g. from 50° C. to 80° C., more preferably 55° C.-75° C.

The specific solvent system used in the invention comprises one or more alcohols, such as at least one alcohol having 1 to 3 carbon atoms (e.g., methanol, ethanol, isopropyl alcohol, 1-propanol), water and acetone, in a proportion of preferably not less than 70 wt %, more preferably not less than 80 wt %. The solvent that may be contained in addition to alcohol having 1 to 3 carbon atoms, water and acetone is not subject to any particular limitation, and may be any as long as it is miscible with these solvents and is not reactive with citalopram hydrobromide. Examples thereof include toluene, xylene, tetrahydrofuran, diisopropyl ether, 1,2-dimethoxyethane, diethylene glycol dimethyl ether and the like. These may be used alone or in a combination of two or more solvents, wherein the boiling point thereof may be higher than the above-mentioned heating temperature. Examples of the above-mentioned specific solvent system include those containing at least one member selected from the group consisting of methanol, ethanol, isopropyl alcohol and 1-propanol in a proportion of not less than 80 wt %, particularly preferably those containing methanol and isopropyl alcohol in a proportion of not less than 90 wt %.

Addition of a see crystal may be at a temperature of from 30° C. to 48° C., or from 40° C to 48° C.

After the addition of the seed crystal, the crystallization of the salt of citalopram is preferably completed after holding or aging at a temperature below the final cooling temperature (preferably −10° C. to 30° C., more preferably 0° C. to 20° C.) for from 5 minutes to 6 hours, in certain embodiments from 15 minutes to 4 hours, or from 30 minutes to 48 hours, or from 15 minutes to 4 hours, or from 30 minutes to 2 hours.

Alternatively, the step of dissolving by heating may be repeated, so that after the first step of cooling the solution to obtain crystallization, the solution is heated to dissolve a part of the obtained crystals. Thereafter, the solution is again cooled to recrystallize the salt of citalopram while controlling the cooling rate. In this method, the heating/dissolving step is repeated for a portion of the crystals obtained from the first crystallization.

In the following, the invention is illustrated by way of examples. However, the examples are merely intended to illustrate the invention and should not be construed as limiting.

Solid Unit Dosage Forms

In one embodiment, the invention is directed to a tablet prepared by direct compression of a mixture of citalopram base or a pharmaceutically acceptable salt thereof and pharmaceutically acceptable excipients.

In another embodiment, the present invention relates to a solid unit dosage form comprising pharmaceutically acceptable salts of citalopram in crystals with a median particle size of at least 40 μm, preferably in the range of 40-200 μm, even more preferred 45-150 μm, and most preferred 50-100 μm.

Flow, segregation and demixing properties and, the suitability of the citalopram crystals for direct compression depend, besides the median particle size, on the particle side distribution.

The solid unit dosage form according to the invention may contain 2-60% w/w active ingredient calculated as citalopram base, preferably 10-40% w/w active ingredient calculated as citalopram base, and more preferred 15-25% w/w active ingredient calculated as citalopram base. Suitably, the solid unit dosage form of the invention contains 20% w/w active ingredient calculated as citalopram base.

In preferred embodiments, the solid unit dosage form comprisese an active ingredient of citalopram hydrobromide or citalopram hydrochloride. Preferably the active ingredient is citalopram hydrobromide.

The solid unit dosage form according to the invention may also contain a filler selected from lactose, or other sugars e.g. sorbitol, mannitol, dextrose and sucrose, calcium phosphates (dibasic, tribasic, hydrous and anhydrous), starch, modified starches, microcrystalline cellulose, calcium sulphate and/or calcium carbonate. In a preferred embodiment, the solid unit dosage form of the invention does not contain lactose. Preferably the solid unit dosage forms according to the invention do not contain a binder.

The direct compression of citalopram, a filler and other pharmaceutically acceptable excipients into tablets has the great advantage, that the granulation and a drying step is avoided. Further, as the granulation step is avoided, it is no longer necessary to add a binding agent.

One suitable filler is a microcrystalline cellulose, such as ProSolv SMCC90, manufactured by Penwest Pharmaceuticals, or Avicel PH 200, manufactured by FMC Corporation.

Besides the active ingredient and filler, the solid pharmaceutical unit dosage forms may include various other conventional excipients such as disintegrants, and optionally minor amounts of lubricants, colorants, and sweeteners.

Lubricants used according to the invention may suitably be one or more of the following metallic stearates (magnesium, calcium, sodium), stearic acid, wax, hydrogenated vegetable oil, talc and colloidal silica. Suitably the lubricant is magnesium stearate or calcium stearate.

Disintegrants include sodium starch glycolate, croscarmellose, crospovidone, low substituted hydroxypropylcellulose, modified cornstarch, pregelatizined starch and natural starch.

The solid, pharmaceutical unit dosage form of the invention may be prepared by conventional methods using a tablet press with forced feed capability.

### EXAMPLE 1

Crystallisation of Citalopram Hydrobromide into Large Crystals

Citalopram hydrobromide (200 g) is dissolved in a mixture of methanol (200 g) and water (20 g) at 69° C. The solution is cooled down to 30° C., seeded with citalopram hydrobromide crystals and kept at 30° C. for 24 hours, whereupon it is cooled down to 10° C. within 1 hour. The crystals are isolated by filtration, washed with cold methanol and dried. The particle size distribution for the resulting crystals is listed in table 1.

### EXAMPLE 2

Crystallisation of Citalopram Hydrobromide into Large Crystals

Citalopram hydrobromide (12.0 kg) is dissolved in a mixture of methanol (12.5 kg) and water (1.2 kg) at reflux. The solution is cooled down to 30° C., seeded with citalopram hydrobromide crystals (27 g) and kept at 30° C. for 16 hours, whereupon it is cooled down to 10° C. within 1 hour. The crystals are isolated by filtration, washed with cold (10° C.) methanol (3.5 kg) and dried. The particle size distribution for the resulting crystals is listed in table 1.

### EXAMPLE 3

Crystallisation of Citalopram Hydrobromide into Small Crystals

Citalopram hydrobromide (200 kg) is dissolved in a mixture of methanol (170 L) and acetone (680 L) at 56° C. The solution is cooled down to 15° C., seeded with citalopram hydrobromide crystals (50 g), hexane (1600 L) is gradually added within 60 minutes, whereupon the suspension is left standing with moderate stirring and cooling for 8 hours. The crystals are isolated by filtration, washed first with a cold (10° C.) mixture of acetone (50 L) and hexane then with cold (10° C.) hexane (220 L) and dried. The particle size distribution for the resulting crystals is listed in Table 1.

### EXAMPLE 4

Crystallisation of Citalopram as the Free Base

Citalopram hydrobromide (101 g) is suspended in water (500 tn. L) and toluene (500 mL). NaOH (60 mL, 5 N (ad)) is added and the mixture (pH>10) is stirred for 15 min before the phases are separated. The organic phase is washed with water (2×100 mL) and filtered through a pad of filter help. The volatiles are removed in vacuo and the title compound is obtained as ail oil, n-Heptane (400 mL,) is added and the mixture is heated to 70° C. On cooling, crystals forms. The white crystals of citalopram base are filtered off and dried at ambient temperature over night in vacuo.

TABLE 1
Particle size distribution (Sympatec Helos) for citalopram
hydrobromide crystals and ProSolv SCMC90

| Quantile | Example 1 | Example 2 | Example 3 | ProSolv |
|---|---|---|---|---|
| (%) | (μm) | (μm) | (μm) | SCMC90 (μm) |
| 95 | 465.43 | 549.42 | 96.96 | 279.94 |
| 90 | 342.89 | 352.23 | 72.27 | 231.66 |
| 50 | 96.87 | 52.70 | 14.04 | 114.17 |
| 10 | 16.54 | 11.97 | 1.19 | 32.10 |
| 5 | 8.23 | 6.67 | 0.82 | 20.56 |

### EXAMPLE 5

Tablet Prepared by Direct Compression of Small Citalopram Hydrobromide Crystals

Tablet ingredients:

| | | |
|---|---|---|
| citalopram, HBr | 5800 g | (20% w/w) |
| Prosolv SMCC90 | 23055 g | (79.5% w/w) |
| Magnesium stearate | 145 g | (0.5,% w/w) |

Citalopram hydrobromide crystals from example 3 and ProSolv SMCC90 were blended at 7 rpm for 10 min in a 100 litre Bohle PTM 200 mixer. Magnesium stearate was added and blending continued for 3 min.

25 kg of the resulting mixture was tabletted (125.000 tablets/hour) on a 30 station Fette P 1200/1C tablet press fitted with oblong, embossed, scored 5,5×8 mm punches. Tablet core weight was set to 125 mg. The nominal yield was 200.000 tablets. The tablet press was run until the mixture level was just above the forced feeder, i.e. the tabletting was continued as long as possible in order to identify possible segregation tendencies in the last quantities of mixture.

Tablet Properties:

Diametrical crushing strength: 70 N

Disintegration time: 30 seconds

Friability: NA

Weight variation: 0.84% relative standard deviation (measured on 20 tablets)

Punch adhesion: None observed

Citalopram Content in the Composition During Compression.

Tablets were sampled throughout the compression in order to measure segregation tendency. Since there is a significant size difference between the active ingredient, citalopram hydrobromide, and the inert filler, ProSolv SMCC90, as seen in Table 1, it would be expected that the unequally sized components would segregate, i.e. de-mix, during transfer from blending vessel to tablet press hopper or sitting in the tablet press hopper during tabletting.

Sampling was performed 50 times at regular intervals during tabletting, corresponding to sampling at every 4000 tablets produced. Two tablets were withdrawn for each sample.

The tablets were assayed by a validated method using UV-absorption in an aqueous solution, thus analysing in total 100 tablets. The relative standard deviation in citalopram content was 1.6%

The variability in tablet strength is surprisingly low in view of the small particle size of citalopram hydrobromide as compared to the inert filler.

One possible explanation for this surprising and beneficial result may be that the tendency to segregation between small citalopram crystals said larger filler particles is uniquely balanced by the poor flow properties of the small crystals.

### EXAMPLE 6

Tablet Prepared by Direct Compression of Large Citalopram Hydrobromide Crystals.

Tablet ingredients:

| | |
|---|---|
| citalopram, HBr | (20% w/w) |
| Prosolv SMCC90 | (79.5% w/w) |
| Magnesium stearate | (0.5% w/w) |

Citalopram Hydrobromide Crystals from Example 2 and ProSolv SMCC90 were Blended.

Magnesium Stearate was Added and Blending Continued.

Tablets (125 mg nominel weight) were produced.

The tablets had satisfactory technical properties.

### EXAMPLE 6

Tablet Prepared by Direct Compression of Citalopram Crystals.

Tablet ingredients:

| | |
|---|---|
| citalopram base | (1.6% w/w) |
| ProSolv SMCC90 | (83.3% w/w) |
| Magnesium stearate | (0.7% w/w) |

Citalopram base crystals from example 4 were sieved through sieve aperture of 0.3 ml and mixed with ProSolv SMCC90 for 3 minutes in a Turbula mixer. Magnesium stearate was added and blending continued for 30 seconds.

Tablets were produced on a single punch tabletting machine Korsch EKO.

Tablet Properties:

Tablet strength, mg: 20

Nominel tablet weight, mg: 125

Tablet diameter, mm: 7

Tablet shape: Film coating, special doomed

Diametrical crushing strength: 61.6 N

Disintegration time, min: <1

Friability: 0.1

Mean tablet weight: 125.4

Weight variation: 0.22% relative standard deviation

The tablets produced had satisfactory technical properties.

What is claimed is:

1. Crystals of a pharmaceutically acceptable salt of citalopram a median particle size of at least 40 μm.

2. The crystals of claim 1 which are crystals of citalopram hydrobromide or citalopram hydrochloride.

3. The crystals of claim 2 which are crystals of citalopram hydrobromide.

4. The crystals of claim 3 wherein up to 5% of the crystals have a particle size of less than 8.23 μm.

5. The crystals of claim 3 wherein up to 5% of the crystals have a particle size of less than 6.67 μm.

6. The crystals of claim 3 wherein up to 5% of the crystals have a particle size of less than 0.82 μm.

7. The crystals of claim 3 wherein up to 10% of the crystals have a particle size of less than 16.54 μm.

8. The crystals of claim 3 wherein no more than 10% of the crystals have a particle size of less than 11.97 μm.

9. The crystals of claim 3 wherein up to 10% of the crystals have a particle size of less than 1.19 μm.

10. The crystals of claim 3 wherein up to 50% of the crystals have a particle size of less than 40 μm.

11. Citalopram hydrobromide crystals containing crystals having a particle size of less than 5 μm in a proportion of 35% at most.

12. The citalopram hydrobromide crystals of claim 11, which comprise crystals having a particle size of not less than 20 μm in a proportion of not less than 10%.

13. The citalopram hydrobromide crystals of claim 11, which have an average aspect ratio of not less than 2.0 and not more than 9.0.

14. The citalopram hydrobromide crystals of claim 11, which have an average aspect ratio of not less than 2.5 and less than 4.5.

15. The citalopram hydrobromide crystals of claim 11, which have an average aspect ratio of not less than 4.5 and not more than 6.0.

16. Citalopram hydrobromide crystals having an average aspect ratio of not less than 2.0 and not more than 9.0.

17. Citalopram hydrobromide crystals having an average aspect ratio of not less than 2.5 and less than 4.5.

18. Citalopram hydrobromide crystals having an average aspect ratio of not less than 4.5 and not more than 6.0.

19. A method of crystallizing citalopram hydrobromide, which comprises the steps of
(a) dissolving citalopram hydrobromide in a solvent system comprising one or more alcohols at a temperature between about 50° C. and the refluxing temperature of the solvent system to form a solution, and
(b) cooling the solution to crystallize citalopram hydrobromide while controlling the temperature of the solution.

20. The method of claim 19, wherein said controlling step comprises maintaining the temperature of the solution between 20° C. and 40° C.

21. The method of claim 19, wherein said controlling step comprises maintaining the temperature of the solution between 25° C. and 35° C.

22. The method of claim 19, which comprises controlling the cooling rate of the solution in a temperature range of from 20° C. to 40° C. at an average cooling rate of 20° C./hour.

23. The method of claim 16, which comprises cooling the solution at an average rate of 20° C. per hour.

24. A method for crystallizing citalopram hydrobromide, which comprises the steps of dissolving, by heating, citalopram hydrobromide in a solvent comprising at least one member selected from the group consisting of alcohol having 1 to 3 carbon atoms, water and acetone and
(B1) cooling the resulting product to allow for crystallization while controlling a cooling rate.

25. The method of claim 23, which comprises controlling the cooling rate of the solution in a temperature range of from 0° C. to 80° C.

26. The method of claim 23, which comprises controlling an average cooling rate of the solution in the temperature range of from 20° C. to 40° C. to not less than 30° C./hour and not more than 60° C./hour.

27. The method of claim 23, which comprises controlling an average cooling rate of the solution in a temperature range of from 20° C. to 40° C. to not less than 0.5° C./hour and less than 30° C./hour.

28. The method of claim 23, which comprises, after cooling to a temperature range of from not less than 30° C. to less than 48° C., adding a seed crystal of citalopram hydrobromide for crystallization.

29. A method for crystallizing citalopram hydrobromide, which comprises the steps of
(A2) dissolving, by heating, citalopram hydrobromide in a solvent comprising at least one member selected from the group consisting of alcohol having 1 to 3 carbon atoms, water and acetone,
(B2) cooling the obtained solution to achieve crystallization,
(C2) dissolving a part of the obtained crystals by heating, and
(D2) recrystallizing while controlling a cooling rate.

30. The method according to claim 28, which comprises cooling to a temperature range of from not less than 30° C. to less than 48° C. in (B2).

31. The method according to claim 28, which comprises, after cooling to a temperature range of from not less than 30° C. to less than 48° C., adding a seed crystal of citalopram hydrobromide for crystallization in (B2).

32. The method according to claim 28, which comprises dissolving a part of the crystals by heating to not less than 48° C. and not more than 60° C. in (C2).

33. The method according to claim 28, which comprises controlling the average cooling rate of the solution in the temperature range of from (the heating temperature in (C2)) to (said heating temperature −30° C.) to not less than 30° C./hour and not more than 90° C./hour in (D2.).

34. The method according to claim 28, which comprises controlling the average cooling rate of the solution in the temperature range of from (the heating temperature in (C2)) to (said heating temperature −30° C.) to not less than 1° C./hour and less than 30° C./hour in (D2).
